(19) United States
(12) Patent Application Publication
(10) Pub. No.: US 2016/0250253 A1
(43) Pub. Date: Sep. 1, 2016

(54) STRONTIUM-CONTAINING COMPLEXES FOR TREATING GASTROESOPHAGEAL REFLUX AND BARRETT'S ESOPHAGUS

(71) Applicant: COSMEDERM BIOSCIENCE, INC., San Diego, CA (US)

(72) Inventor: Gary S. HAHN, San Diego, CA (US)

(21) Appl. No.: 15/023,974

(22) PCT Filed: Sep. 24, 2014
(86) PCT No.: PCT/US14/57244

§ 371 Date: Mar. 22, 2016

Related U.S. Application Data
(60) Provisional application No. 61/881,850, filed on Sep. 24, 2013.

(51) Int. Cl. A61K 33/00; A61K 31/734; A61K 31/191; A61K 31/198; A61K 33/08; A61K 31/4164; A61K 31/4184; A61K 31/79; A61K 33/10

(52) U.S. Cl. CPC A61K 33/00; A61K 31/79; A61K 31/734; A61K 31/191; A61K 33/10; A61K 33/08; A61K 31/4164; A61K 31/4184; A61K 31/198

(57) ABSTRACT

Therapeutically-active compositions for treating pain, irritation, and inflammation associated with gastroesophageal reflux and Barrett's esophagus that combines at least one acid neutralizing strontium salt with at least one polyhydroxyphenol, at least one polymer, and optionally at least one cysteine based antioxidant.

### CROSS-REFERENCE TO RELATED APPLICATIONS

This application claims priority to U.S. provisional application No. 61/881,850, filed on Sep. 24, 2013, the contents of which are incorporated herein by reference in their entirety.

### TECHNICAL FIELD

The present disclosure consists of therapeutically-active compositions for treating pain, irritation, and inflammation associated with gastroesophageal reflux and Barrett's esophagus that combines, for example, at least one acid neutralizing strontium salt with at least one polyhydroxyphenol, at least one polymer, and optionally at least one cysteine based antioxidant.

### BACKGROUND

Gastroesophageal reflux (GER), commonly referred to as heartburn, is the movement of acidic material from the stomach back into the esophagus. Common symptoms of GER include, but are not limited to, burning-type pain in the lower part of the mid-chest, behind the breast bone, and in the mid-abdomen, sore throat, coughing, and water brash, the sensation of a bitter taste in the mouth. GER is usually a temporary condition that causes no lasting damage to the affected tissues. However, in some individuals, chronic bouts of GER leads to severe damage to the esophageal lining, a condition referred to as gastroesophageal reflux disease (GERD). If GERD is left untreated, the continual damage to the esophageal lining can develop into Barrett's esophagus, a precancerous condition that can lead to esophageal cancer.

The American Gastrointestinal Endoscopic Surgeons (SAGES) estimates that 36-77% of men and women have symptoms of GERD. Of this population, between 15-50% have heartburn symptoms every month, 14-20% have symptoms weekly and 7% have heartburn symptoms daily.

Barrett's esophagus, sometimes called Barrett's syndrome or columnar epithelium lined lower esophagus (CELLO), refers to an abnormal change (metaplasia) in the cells of the lower portion of the esophagus. Specifically, the esophageal tissue changes into tissue that is similar to intestinal lining. The main cause of Barrett's esophagus is believed to be an adaptation to chronic acid exposure from acid reflux. Barrett's esophagus is found in 5-10% of patients who seek medical care for GER. People with Barrett's esophagus are at an increased risk for developing esophageal adenocarcinoma.

The high prevalence of GER has resulted in the development of a multibillion dollar treatment industry in which prescription and over the counter (OTC) drugs are among the world's largest selling category. The majority of drugs approved for GER treatment are designed to reduce the acidity of the stomach's contents either by reducing hydrochloric acid secretion by the stomach's parietal cells via histamine H2 receptor antagonists (H2RA) and proton pump inhibitors or by chemically neutralizing the acid by using carbonate or hydroxide-containing antacids

### SUMMARY

In accordance with the teachings herein, the present disclosure relates generally to compositions of at least one acid neutralizing strontium salt, at least one polyhydroxyphenol, at least one polymer, and optionally at least one cysteine based antioxidant for use in treating and preventing symptoms associated with gastroesophageal reflux (GER) and Barrett's esophagus. The compositions provide a new therapeutic approach to treating GER and Barrett's esophagus by using a combination of ingredients that reduce the pain and inflammation by blocking the acid and inflammation-inducing biochemical pathways at multiple points and thereby provide and overall therapeutic benefit far greater than any individual ingredient. A particularly advantageous property of this disclosure is that each of the ingredients that provide therapeutic benefits in the present disclosure (e.g. strontium, gallic acid and cystine as one exemplary ingredient combination) are found in foods that are consumed every day in typical meals and for this reason lack the potential adverse reactions that can occur with the use of synthetic ingredients like histamine H2 receptor antagonists and proton pump inhibitors. Since most GER patients must consume a GER treatment, and for some patients must consume daily for their lifetime, the inherent safety of the ingredients used in the present disclosure provide a substantial medical benefit over the most commonly used conventional GER treatments.

In one embodiment are compounds for treating GER, GERD and Barrett's esophagus that include three different components: at least one acid neutralizing strontium salt selected from the group consisting of strontium carbonate, strontium bicarbonate, strontium hydroxide, strontium phosphate, and strontium citrate; at least one polyhydroxyphenol selected from the group consisting of gallic acid, tannic acid, pentagalloyl glucose, ellagic acid, and esters thereof; and at least on polymer selected from the group consisting of alginic acid, polyvinylpyrrolidone, and xanthan gum. In an alternate embodiment, the polymer is a mixture of alginic acid and polyvinylpyrrolidone. In another embodiment, the strontium salt is strontium carbonate. In another embodiment, the strontium salt is strontium bicarbonate. In another embodiment, the strontium salt is strontium hydroxide. In another embodiment, the strontium salt is strontium phosphate. In another embodiment, the strontium salt is strontium citrate. In another embodiment, the pholyhydroxyphenol is gallic acid. In another embodiment, the pholyhydroxyphenol is tannic acid. In another embodiment, the pholyhydroxyphenol is pentagalloyl glucose. In another embodiment, the pholyhydroxyphenol is ellagic acid. In another embodiment, the polymer is alginic acid. In another embodiment, the polymer is polyvinylpyrrolidone. In another embodiment, the polymer is xanthan gum. In another embodiment, the polymer is a combination of alginic acid and polyvinylpyrrolidone. In another embodiment, the strontium salt is strontium carbonate, the polyhydroxyphenol is gallic acid and the polymer is a combination of alginic acid and polyvinylpyrrolidone. In another embodiment, the strontium salt is strontium carbonate, the polyhydroxyphenol is tannic acid and the polymer is a combination of alginic acid and polyvinylpyrrolidone. In another embodiment, strontium salt is strontium carbonate, the polyhydroxyphenol is pentagalloyl glucose and the polymer is a combination of alginic acid and polyvinylpyrrolidone. In another embodiment, the strontium salt is strontium carbonate, the polyhydroxyphenol is ellagic acid and the polymer is a combination of alginic acid and polyvinylpyrrolidone.

In another embodiment are compounds for treating GER, GERD, and Barrett's esophagus that include four different components: at least one acid neutralizing strontium salt selected from the group consisting of strontium carbonate, strontium bicarbonate, strontium hydroxide, strontium phosphate, and strontium citrate; at least one polyhydroxyphenol selected from the group consisting of gallic acid, tannic acid, pentagalloyl glucose, ellagic acid, and esters thereof; at least one cysteine based antioxidant selected from the group consisting of L-cystine and N,S-diacetylcysteine; and at least one polymer selected from the group consisting of alginic acid, polyvinylpyrrolidone, and xanthan gum. In another alternate embodiment, the polymer is a mixture of alginic acid and polyvinylpyrrolidone. In another embodiment, the strontium salt is strontium carbonate. In another embodiment, the strontium salt is strontium bicarbonate. In another embodiment, the strontium salt is strontium hydroxide. In another embodiment, the strontium salt is strontium phosphate. In another embodiment, the strontium salt is strontium citrate. In another embodiment, the polyhydroxyphenol is gallic acid. In another embodiment, the polyhydroxyphenol is tannic acid. In another embodiment, the polyhydroxyphenol is pentagalloyl glucose. In another embodiment, the polyhydroxyphenol is ellagic acid. In another embodiment, the cysteine based antioxidant is cystine. In another embodiment, the cysteine based antioxidant is N,S-diacetylcysteine. In another embodiment, the polymer is alginic acid. In another embodiment, the polymer is polyvinylpyrrolidone. In another embodiment, the polymer is xanthan gum. In another embodiment, the polymer is a combination of alginic acid and polyvinylpyrrolidone. In another embodiment, the strontium salt is strontium carbonate, the polyhydroxyphenol is gallic acid, the cysteine based antioxidant is cystine, and the polymer is a combination of alginic acid and polyvinylpyrrolidone. In another embodiment, the strontium salt is strontium carbonate, the polyhydroxyphenol is gallic acid, the cysteine based antioxidant is N,S-diacetylcysteine, and the polymer is a combination of alginic acid and polyvinylpyrrolidone. In another embodiment, the strontium salt is strontium carbonate, the polyhydroxyphenol is tannic acid, the cysteine based antioxidant is cystine, and the polymer is a combination of alginic acid and polyvinylpyrrolidone. In another embodiment, the strontium salt is strontium carbonate, the polyhydroxyphenol is tannic acid, the cysteine based antioxidant is N,S-diacetylcysteine, and the polymer is a combination of alginic acid and polyvinylpyrrolidone. In another embodiment, the strontium salt is strontium carbonate, the polyhydroxyphenol is pentagalloyl glucose, the cysteine based antioxidant is cystine, and the polymer is a combination of alginic acid and polyvinylpyrrolidone. In another embodiment, the strontium salt is strontium carbonate, the polyhydroxyphenol is pentagalloyl glucose, the cysteine based antioxidant is N,S-diacetylcysteine, and the polymer is a combination of alginic acid and polyvinylpyrrolidone. In another embodiment, the strontium salt is strontium carbonate, the polyhydroxyphenol is ellagic acid, the cysteine based antioxidant is cystine, and the polymer is a combination of alginic acid and polyvinylpyrrolidone. In another embodiment, the strontium salt is strontium carbonate, the polyhydroxyphenol is ellagic acid, the cysteine based antioxidant is N,S-diacetylcysteine, and the polymer is a combination of alginic acid and polyvinylpyrrolidone.

In another embodiment, the aforementioned compositions further comprise at least one additional pharmaceutical agents known for treating GER are added. In one embodiment, the pharmaceutical agent is an acid reducer such as calcium carbonate, sodium bicarbonate, sodium citrate, aluminum hydroxide, aluminum phosphate, magnesium hydroxide, magnesium carbonate, magaldrate, almagate, or hydrotalcite. In another embodiment, the pharmaceutical agent is a histamine H2 receptor antagonist such as cimetidine, famotidine, nizatidine, or rantidine. In yet another embodiment, the pharmaceutical agent is a proton pump inhibitor such as omeprazole, lansoprazole, rabeprazole, pantoprazole, or esomeprazole. In yet another embodiment, combinations of the above mentioned pharmaceutical agents are added.

In another embodiment, the aforementioned compositions further comprise at least one additional acid neutralizing agent. In another embodiment, the acid neutralizing agent is selected from the group consisting of calcium carbonate, sodium bicarbonate, sodium citrate, aluminum hydroxide, aluminum phosphate, magnesium hydroxide, magnesium carbonate, magaldrate, almagate, and hydrotalcite. In another embodiment, the acid neutralizing agent is calcium carbonate. In another embodiment, the acid neutralizing agent is sodium bicarbonate. In another embodiment, the acid neutralizing agent is sodium citrate. In another embodiment, the acid neutralizing agent is aluminum hydroxide. In another embodiment, the acid neutralizing agent is aluminum phosphate. In another embodiment, the acid neutralizing agent is magnesium hydroxide. In another embodiment, the acid neutralizing agent is magnesium carbonate. In another embodiment, the acid neutralizing agent is magaldrate. In another embodiment, the acid neutralizing agent is almagate. In another embodiment, the acid neutralizing agent is hydrotalcite. In another embodiment, the acid neutralizing agent is a mixture of aluminum hydroxide and magnesium hydroxide.

In another embodiment, the aforementioned compositions further comprise a histamine H2 receptor antagonist. In another embodiment, the histamine H2 receptor antagonist is selected from the group consisting of cimetidine, famotidine, nizatidine, and rantidine. In another embodiment, the histamine H2 receptor antagonist is cimetidine. In another embodiment, the histamine H2 receptor antagonist is famotidine. In another embodiment, the histamine H2 receptor antagonist is nizatidine. In another embodiment, the histamine H2 receptor antagonist is rantidine.

In another embodiment, the aforementioned compositions further comprise a proton pump inhibitor. In another embodiment, the proton pump inhibitor is selected from the group consisting of omeprazole, lansoprazole, rabeprazole, pantoprazole, and esomeprazole. In another embodiment, the proton pump inhibitor is omeprazole. In another embodiment, the proton pump inhibitor is lansoprazole. In another embodiment, the proton pump inhibitor is rabeprazole. In another embodiment, the proton pump inhibitor is pantoprazole. In another embodiment, the proton pump inhibitor is esomeprazole.

In another embodiment, the aforementioned compositions are formulated as a solid. In another embodiment, the solid is a lozenge, swallowable tablet, chewable tablet, effervescent tablet, or chewing gum. In another embodiment, the aforementioned compositions are formulated as a liquid.

In another embodiment is a method of treating symptoms associated with GER, GERD, or Barrett's esophagus in a patient. The patient is treated by administering the above described formulations. In an alternate embodiment, the patient is treated proactively by administering the formulations before the onset of symptoms, for example, taking the formulation within an hour or within 30 minutes after a meal or with the meal or before the meal. In yet another alternative embodiment, the patient is treated by administering the formulation after the presentation of gastroesophageal reflux symptoms.

### DETAILED DESCRIPTION

The present disclosure relates to compounds for use in treating symptoms associated with gastroesophageal reflux (GER), gastroesophageal reflux disease (GERD), Barrett's esophagus and other related conditions (all referred to as “GER” herein unless otherwise indicated).

To block the pain and inflammation associated with GER, conventional treatments use a single active ingredient that blocks a single biochemical pathway. For example, proton pump inhibitors selectively block the hydrogen/potassium ATPase that uses the energy of ATP to translocate protons into the stomach and thus reduce the pH of its contents. Similarly, histamine H2 receptor antagonists (H2RA) selectively block the H2 histamine receptor that must be activated by histamine to stimulate proton secretion into the stomach. Such single receptor targeting approach typically requires that the regulatory molecule being used be very potent and have the ability to block most of the target's biochemical activity in order to achieve the desired pharmacological benefits. While blockade of the target receptor in the target organ, the hydrogen/potassium ATPase in the case of a proton pump inhibitor in the acid-secreting parietal cell of the stomach, it is not uncommon to have blockade of the same or similar molecular target in other organs that produces adverse reactions. Such is the case with proton pump inhibitors since they also block a similar target receptor in bones that causes bone weakening and ultimately osteoporosis.

The present disclosure presents a different pharmacological approach that can achieve both high efficacy and substantially reduce adverse reactions. Instead of using a single highly potent drug to block acid secretion that will indirectly reduce pain and inflammation, the present disclosure uses multiple relatively low potency ingredients that block critical pain and inflammation pathway regulatory proteins and multiple points in each pathway. The result is partial inhibition of a particular regulatory protein, thus allowing it to function at a reduced level and continue to regulate other parallel pathways that are not inflammation and pain generators. The net effect on the pathway will be substantially greater due to the multiple levels of inhibition. By preserving the activity of partially blocked regulatory protein, it can continue to participate in the regulation of other pathways and thereby reduce the potential for toxic or adverse reactions. In a metaphoric sense, this is akin to turning down the volume of a radio instead of shutting it off completely.

The present disclosure optimally uses multiple ingredients that reduce acid-induced pain and inflammation by different mechanisms. Additionally each ingredient partially inhibits one or more pathways inhibited by one or more of the other ingredients, thus providing a unique synergy of therapeutic activity.

In one embodiment, the compounds consist of therapeutically-active compositions that combine at least one acid neutralizing strontium salt with at least one polyhydroxyphenol and at least one polymer. In another embodiment, the compounds consist of therapeutically-active compositions that combine at least one acid neutralizing strontium salt with at least one polyhydroxyphenol, at least one cysteine based antioxidant, and at least one polymer. The combination increases the overall therapeutic potency of the combination beyond the potency of any of the separate constituents. Specifically, the combinations described herein perform several functions over an extended period of time: (1) increase the pH of the stomach contents, (2) block stomach acid production, (3) create a protective barrier in the esophagus, (4) create a protective barrier in the stomach (5) reduce pain, and (6) reduce inflammation. The compounds and their effects on pain, inflammation, and acid production pathways are discussed in greater detail below.

In the description that follows, a number of terms are extensively utilized. In order to provide a clear and consistent understanding of the specification and claims, including the scope to be given such terms, the following non-limiting definitions are provided.

When the terms “one,” “a,” or “an” are used in this disclosure, they mean “at least one” or “one or more,” unless otherwise indicated.

The term “epithelium” refers to external surfaces of the body in the broadest sense of the word and therefore implicitly includes all keratinized skin as well as mucous membranes, for example, the mouth, throat, surfaces of the eye, the respiratory tract, and the gastrointestinal tract.

The term “gastroesophageal reflux” or “GER” refers to the movement of contents from the stomach back into the esophagus and mouth. The term also includes other names such as pyrosis, dyspepsia, heartburn, acid reflux, and acid indigestion. Unless otherwise indicated, the use of GER in the present disclosure includes GERD (defined below) and Barrett's esophagus.

The term “gastroesophageal reflux disease” or “GERD” refers to acute and chronic inflammation and damage to the esophagus due to repetitive exposure to stomach contents. The term also includes other names such as gastro-oesophageal reflux disease (GORD), gastric reflux disease, and acid reflux disease.

The term “complex” as used herein refers to a combination of at least one acid neutralizing strontium salt, at least one polyhydroxyphenol, at least one polymer, and optionally at least one cysteine based antioxidant. Additional therapeutic agents may be added to the complex such as acid reducers, proton pump inhibitors, histamine H2 receptor antagonist, or other pharmaceutical excipients known in the art.

The term “acid neutralizing” as used herein refers to a chemical that is capable of reducing the acidity of a liquid. The chemical does not need to be able to bring the acid to a complete neutral state. Non-limiting examples of acid neutralizing chemicals include salts of carbonates, bicarbonates, hydroxides, phosphates, and citrates.

### Nociception and Inflammation Pathways

Nociception is the neural processes of encoding and processing stimuli that have the potential to damage tissue. Nociceptors are specialized nerves located throughout the body that detect mechanical, thermal or chemical changes. There are two classes of nociceptors, the first class is “A-delta” nerves, which respond to physical trauma by transmitting a pain sensation with a sharp, pricking quality. The second class is “Type C” nerves (TCN), which are chemical sensors that respond to irritants from our environment, such as microbes, temperature extremes, and ionizing radiation and transmit diffuse sensations of burning pain, stinging pain or itching (“irritation”). When excessively stimulated, TCN can also release neuropeptides (e.g., Substance P) that directly activate histamine-containing mast cells and attract and activate other immune system cells such as neutrophils that cause redness, swelling and even local tissue damage. After activation by a stimuli, nociceptors synapse near the spinal cord in the dorsal root ganglia (DRG) and release neurotransmitters that activate nerve pathways that relay signals to the brain. The brain interprets the signals as various types of pain or itch.

### A. Acute, Chronic, and Neuropathic Pain and Pruritus Occur Upon Nociceptor Activation

Exposure to stimuli activates nociceptors. Depending on the stimuli, both types of nociceptors may be activated or in many instances either the A-delta or TCN are preferentially activated. Since only the TCNs extend to the outermost portions of the body, such as the skin, mouth, nose, throat, eyes, etc. (herein referred to as “epithelium”) and may be activated by virtually any process that changes the local biochemistry of the epithelium, TCNs are preferentially activated in response to most irritating stimuli. Upon activation of TCNs, the TCNs transmit a signal to the spinal cord and trigger neurotransmitter release in the DRG that activate nerves in the spinal cord that relay the pain and itch signals to the brain. Acute activation of TCNs that is caused by exposure to a chemical irritant, such as stomach acid, typically causes painful or pruritic sensations that last only several days and is termed “nociceptive pain”. When the stimulus is prolonged or excessively severe, painful sensations or pruritus can continue for many years. Such chronic pain or pruritus caused by excessive nociceptor activation or damage is termed “neuropathic” and is among one of the most difficult conditions to treat.

B. Nociceptive Signals are Typically Encoded as Precisely-Timed Changes of Intracellular Calcium Concentration that Travel as “Calcium Waves” within Nociceptors

No matter what causes nociceptor activation, the event is encoded into a universal code; a complex change in the intracellular calcium concentration that, in turn, is transmitted throughout the nociceptor. Calcium thus acts as a universal “second messenger” and information transmitted by a nociceptor, including the intensity and quality of pain or pruritus is converted into a language made up of rapidly changing calcium concentrations. Since nerves in general and nociceptors in particular transmit their calcium code typically within about 1/1000^{th} of a second, the timing and spatial distribution of calcium must be exquisitely regulated to accurately transmit the encoded information. In virtually all nerves, including nociceptors, the intensity of the signal (e.g., the severity of pain or pruritus) is encoded as a change in frequency of calcium waves that trigger neurotransmitters that are released into the synapse and activate post-synaptic nerves that relay the information ultimately to the brain. The higher the frequency, the more intense the perceived sensation. When a nociceptor is activated, the calcium signal is transmitted through multiple biochemical pathways, many of which operate in sequence such that the output of one pathway becomes the input of the next.

C. Nociceptive Signals and the Biochemical Pathways that Encode Signals Have an Output that is Logarithmically Related to the Input

The many nociceptor pathways as well as the overall neurotransmitter release by a nociceptor is typically logarithmically related to the intensity of the stimulus. For example, if the irritant caused the nociceptor activation to increase its frequency of activation, also called depolarization, from 10 to 50 per second, the frequency of the resultant neurotransmitter release may only increase by a factor of 1.7 (Log 10=1.0; Log 50=1.7). This fact is particularly relevant since it suggests that a relatively small amount of inhibition of a nociceptor's activation can cause a large reduction in the perceived severity of the painful or pruritic stimulus. Since their are many separate pathways in nociceptors that act in sequence to encode and transmit the irritant stimulus, inhibiting each of the sequential pathways at one or more of a pathway's steps has the potential to produce a very large cumulative reduction of the painful or pruritic sensation.

Recent research has demonstrated that high osmolarity formulations activate specific osmotic sensors present on nociceptors, keratinocytes and immune or inflammatory cells. An example of this is the “salt in the wound” effect that causes stinging and burning if a concentrated solution of a simple salt is poured into wound. In addition to causing discomfort, high osmolarity solutions can directly activate inflammatory cells and cause them to release chemicals that cause nociceptor activation.

### D. The Development and Maintenance of Neuropathic Pain or Pruritus Requires Excessive and Continuous Nociceptor Activation

In order for a neuropathic condition to develop, nociceptors must be continuously activated by a potent stimulus. The duration of the activation required may substantially vary depending on the specific nerve injury or stimulant. When such activation occurs, the peripheral nociceptors that innervate the epithelium and mucous membranes such as those of the esophagus may become sensitized within hours and may continue to increase their sensitivity to irritants and may even be activated by stimuli that are normally not irritating. Release of multiple inflammatory mediators that accompany any trauma or inflammation are also important contributors to sensitization.

In order to establish a neuropathic state, sensory nerves in the DRG that receive sensory input from the TCN must also become sensitized. As for the peripheral TCN, the central neurons require sustained, high intensity activation for an extended period of time that may be as short as several weeks or much longer. The presence of inflammation, infectious agents, or trauma can accelerate the sensitized, neuropathic state. Due to neuronal “cross-talk,” it is common for an initially small painful portion of sensitized tissue, for example, as occurs in post-herpetic neuralgia, to expand to the adjacent tissue via nociceptors that were uninjured, including A-delta nociceptors. Sensitized neuropathic tissue may also generate painful stimuli in response to mechanical pressure, e.g. coughing or swallowing, or temperature changes, a condition known as allodynia.

The sensitized state in both the peripheral nociceptors and their central counterparts is a form of activity-dependent plasticity that is very similar to the neurons in the CNS that form memories. In the case of neuropathic pain or pruritus, the nociceptive response produces a “memory of pain or itching.” The molecules and pathways that produce the long-lasting neuronal sensitization are reasonably well defined. In particular, the activation of intracellular kinases. Of particular importance are protein kinase A and C (PKA and PKC, respectively), each of which exist in several different forms and the mitogen activated protein kinases (MAPK) that include the p38 MAPK, ERK1/2 MAPK and the JNK MAPK. These kinases are activated by a broad range of environmental “danger signals” and internal cytokines and growth factors exposures including ionizing radiation, reactive oxygen species (ROS) always accompany infection and trauma. When activated, these kinases are activated in multiple pathways and give rise to sequential cascades that result in regulation and activation of genes that regulate well over 100 different molecules that activate immune cells, produce inflammation and molecules that influence ion channels, and molecular sensors that cause the peripheral and central nociceptor sensitization that causes neuropathic pain and pruritus. Among these inflammation and immune-system activating genes, the most important is called Nuclear Factor, Immunoglobulin Light Chain Kappa, Enhancer of B Cells, abbreviated NF-Kappa B, called the “Master Gene Regulator of Inflammation.” Additionally, some of these kinases like PKC can directly sensitize and activate nociceptors that cause calcium influx and interfere with strontium's ability to alter the calcium dynamics that occur in neuropathic states.

E. Stimuli that Oxidize Intracellular Glutathione Trigger Multiple Nociceptor-Activating Pathways

Of the many conditions that may cause nociceptor activation during the development of neuropathic conditions, the redox state of a nociceptor can produce some of the most potent acute and chronic nociceptor activating stimuli that exist. One of the most important regulatory signals that cause a cell to convert to a defensive state in which multiple inflammatory and cell protective immune activators are activated is the intracellular ratio of reduced glutathione (GSH) to oxidized glutathione (GSSG). Glutathione is the most plentiful intracellular thiol antioxidant, and is among the most important signal generators that trigger a cell to synthesize powerful inflammatory mediators and activate genes that, in turn, activate virtually every immune system inflammatory cell. The ratio of reduced glutathione, GSH, to the oxidized form, GSSG, is normally 9 to 1 or more. When cells are exposed to trauma, infection, inflammation or inflammatory mediators, ionizing radiation or general “cellular stress,” the amount of reduced glutathione plummets and directly trigger multiple cascades of gene activation that ultimately lead to the synthesis of well over 100 inflammatory mediators, pro-inflammatory cytokines (e.g., TNF-alpha, IL-1, IL-6 and many others), and cytokines that attract and active inflammatory immune cells, all of which sensitize and activate nociceptors that transmit pain and pruritic signals, and in turn amplify these inflammatory cascades by neurogenic inflammatory pathways. Many of a cell's most important regulators of inflammation and immune defense are highly sensitive to a reduction in a cell's GSH concentration, and are directly activated by a low GSH/GSSG ratio indicating that a cell is in an oxidative redox state.

Perhaps the most important of these redox-sensitive regulatory pathways is NF-Kappa B. This molecule is responsible for that directly or indirectly inducing the synthesis of among the most important and powerful inflammation activators, including TNF-alpha and many of the inflammatory interleukins and chemokines that attract inflammatory cells that secrete mediators that directly activate nociceptors and thus increase their long-term sensitization and conversion to a neuropathic state.

Since NF-Kappa B acts as a “final common pathway” for activation of multiple inflammatory pathways, substances that reduce or block NF-Kappa B activation will have substantial and broad anti-inflammatory activity and will block many forms of immune system-mediated activation of inflammatory pathways. NF-Kappa B is also one of the many regulatory molecules that is directly activated by an oxidative intracellular environment—one in which the ratio of reduced glutathione (GSH) to oxidized glutathione (GSSG) is minimized. This oxidative environment directly activates NF-Kappa B that greatly increases the synthesis of nociceptor-activating mediators and cytokines.

Since both peripheral nociceptors with endings in the epithelium and central nociceptors in the DRG and spinal cord become sensitized upon continuous activation, activation of NF-Kappa B is an important and critical stimulator of neuropathic sensitization.

F. Activation of Toll-Like Receptors by Microbes Activate Gene Transcription by NF-Kappa B that Sensitizes Activate Nociceptors

Epidermal cells, mucosal cells and virtually all inflammatory immune cells have many receptors that that can cause nociceptor activation. Among the most important are Toll-Like Receptors (TLRs), molecules that recognize conserved molecular structures of bacteria, fungi and viruses. Upon activation, TLRs trigger multiple inflammatory and nociceptor activating pathways, all of which lead to NF-Kappa B activation.

G. Activation of NF-Kappa B Produces Chemokines that Attract Inflammatory Cells

One of the most important consequences of NF-Kappa B is to stimulate the production of chemokines, including IL-8, that attract and activate neutrophils, a blood-borne white blood cell (WBC) that typically constitutes over 50% of all WBCs in the blood. Neutrophils are the first responders to any type of trauma, infection or inflammatory process and accumulate at the triggering site in massive quantities. Upon activation by IL-8 and other inflammatory mediators, neutrophils produce massive levels of powerful oxidants, reactive oxygen species (ROS; e.g., superoxide, hydrogen peroxide, nitric oxide and hypochlorous acid) that rapidly deplete GSH from cells, including nociceptors, thus promoting oxidative activation of NF-Kappa B and activation of many kinases, including protein kinase A, protein kinase C and mitogen-activated protein kinases that act to amplify virtually all inflammatory pathways that directly activate nociceptors.

Activation of these multiple independent inflammatory pathways and inflammatory cells result in intense activation of nociceptors that contribute to the development of neuropathic sensitization and neuropathic pain and pruritus.

Such activation of nociceptors also causes them to release Substance P that directly triggers mast cell activation and release of histamine, TNF-alpha, IL-1, IL-6, IL-8 and many more inflammatory substances that further activate nociceptors. Due to the simultaneous activation of multiple inflammatory and nociceptor-activating pathways, there is a net amplification of nociceptor activation that is known to directly lead no neuropathic pain and pruritus.

### Cellular and Molecular Pathways Triggered by Stomach Acid Secretion

Unlike the gastric mucosa that is protected from the acid and protease-rich contents of the stomach by multiple levels of cellular and physical barriers, the esophageal mucosa lacks such protection and is easily damaged by reflux of the stomach contents. Such damage allows both hydrochloric acid and the stomach-derived protease, pepsin, to penetrate the esophageal mucosa in high quantities and directly trigger multiple inflammatory and pain-producing pathways. Among these, acid-induced neurogenic inflammation is among the most important.

Biochemical pathways function by the sequential transmission of signals between multiple molecules until a particular endpoint is achieved. In the case of stomach acid secretion, the presence of food molecules in the stomach activate nutrient sensors in stomach cells that trigger a cascade of biochemical events in multiple cells that ultimately lead to acid secretion.

Similar multi-molecular cascades are mast cells and other inflammatory cells that trigger mucosal inflammation in the esophagus and contribute to its pathogenesis. Mast cells populate the mucosa of the esophagus and contribute significantly to the inflammation caused by reflux of the acidic stomach contents into the esophagus. When activated by stomach acid, mast cells release over 50 distinct inflammatory and pain-inducing mediators that trigger activation of other inflammatory cells and directly activate pain sensations in nociceptors, which in turn, release additional mediators that amplify the overall painful inflammatory reaction that is known to cause the signs and symptoms of GER, GERD, and Barrett's esophagus.

### A. Acid Activation of Inflammatory Mediators

Protons produce pain and inflammation, in part, by activating acid sensitive ion channels (ASIC) and transient receptor potential vanilloid receptor-1 (TRPV-1) ion channel, also known as the capsaicin receptor. Both ASIC and TRPV-1 are present on Type C nociceptors. Upon activation, these receptors depolarize the nociceptors that cause two distinct activities: (1) transmission of calcium coded pain signals to the brain, and (2) Release of substance P, neurokinin A, calcitonin gene-related peptide (CGRP) and other neuropeptides and inflammatory mediators that, in turn, initiate multiple inflammatory and pain-triggering cascades in a process called “neurogenic inflammation.” Of these released neuropeptides, substance P is among the most important since in directly activates mast cells and other inflammatory cells to release a myriad of inflammation mediators. Substance P also causes blood vessels to become permeable causing leakage of plasma from the vessels and allowing inflammatory white blood cells to accumulate at the site of inflammation.

### B. Mast Cell Activation

Within the mast cell, acid activates ASIC that open and allow a burst of calcium influx that triggers multiple cascades of pathways, each of which have multiple sequential ‘molecular steps’ that amplify the calcium signal and ultimately result in inflammatory mediator release. Many of these mediators directly activate adjacent sensory neurons, Type C nociceptors, by activating multiple receptors and ion channels that allow calcium influx that are ultimately transduced into the pain sensation of GER.

### Strontium Affects Nociception and Inflammation Pathways

Strontium's unique therapeutic properties are due to its chemical resemblance to calcium, the most important and universal “second messenger” in nerves and in all other cells that regulate virtually all cellular functions. The calcium ion always has two positive charges and its ionic radius is 0.99 angstroms, about the size of a hydrogen atom. Of all the elements, strontium most closely resembles calcium, since it also only exists as a divalent positively-charged ion and has an ionic radius of 1.13 angstroms. For this reason, strontium typically binds to calcium-binding sites and mimics calcium's activity. Most often a strontium-induced response is less potent and may be as low as about 1/1000th as active as calcium, but for certain calcium-dependent activities, strontium has activity that is nearly the same as calcium or in the range of 1/10th to 1/30th as active as calcium. In other calcium-dependent activities, strontium can be more active than calcium. It is strontium's calcium-mimetic activity that enables strontium to produce its many and varied activities. Since calcium is critical for so many cellular functions, if it were strongly inhibited the effects would be toxic to a cell. In contrast, since strontium can typically substitute for calcium, albeit with lower activity, the activity of the calcium-dependent pathway will not be shut down. Instead, the pathway activity will be reduced, similar to turning down the volume control of a radio. Since strontium, in a metaphoric sense, only turns down the volume control of calcium-dependent pathways rather than shutting down such pathways, the chances of significant adverse reactions or toxicity is much reduced compared to a drug that completely blocks a pathway.

### A. Strontium Alters the Dynamics and Spatial Distribution of Calcium Waves

When irritants from chemicals, disease, trauma or other exposures activate receptors on the surface of TCNs that encode the intensity of their response as rapid changes in intracellular calcium concentrations, these changes can occur in less then 1/1000th of a second and produce highly complex “waves” of changing calcium concentration that propagate through the nerve and triggered most, if not all, of the pathways that cause acute, chronic and neuropathic irritation. In addition to the frequency of calcium waves, alterations in the dynamics of calcium concentration change the duration, magnitude and the precise shape of the calcium waveform that alters the coexisting electrostatic field that is a critical regulator of TCN activity. These changes independently activate the release of multiple inflammatory mediators, including prostaglandins (e.g., PGE2), leukotrienes (e.g., LTB4, C4, D4, and E4) and reactive oxygen species (ROS) including superoxide, hydrogen peroxide, hydroxyl radicals, hypochlorous acid and peroxynitrite.

Strontium thus significantly alters the pain and itch sensations encoded within calcium waves present in painful and pruritic neuropathic conditions, and has the effect of garbling the signal and reducing its perceived intensity by the brain. Due to strontium binding to multiple calcium-dependent signaling pathways, strontium significantly alters calcium-encoded signals by multiple independent mechanisms. Some of the calcium-dependent kinases are known to be essential for the development of neuropathic conditions, since their inhibition in animal models can prevent and or reverse established neuropathic conditions.

Strontium is not able to bind effectively to the calcium binding proteins within the cytoplasmic interior of nociceptors that normally remove calcium within less than a millisecond after calcium enters the nociceptor, thus producing a transient increase in calcium concentration that contributes to the precisely-timed calcium waves. Strontium is also much less effectively pumped into and released from a nociceptor's primary calcium storage site, the endoplasmic reticulum (ER). When a nociceptor-activating signal is received, strontium inhibits the calcium-induced calcium release (CICR) pathway that amplifies the calcium signal, and strontium does not have the ability to regulate inositol triphosphate (IP₃)-induced calcium release by acting to inhibit additional calcium release if the concentration of calcium in the cytoplasm is too high.

Once calcium enters a nociceptor during its activation and depolarization, it activates the release of a massive amount of calcium that is stored in the ER by the CICR pathway. This mechanism has the effect of greatly amplifying the amount of calcium that is available to form a wave and to regulate calcium-dependent pathways. Strontium is over a hundred-fold less active than calcium in its ability to induce CICR and thus significantly alters the calcium concentration changes that normally occur in response to irritants. When in the ER, strontium also binds much less avidly to the ER calcium binding proteins that act as buffers and sequester the free calcium until it is released by CICR or other similar mechanisms. As a result, strontium reaches a concentration of more than 150% greater than calcium and displaces calcium from performing its amplifying function during CICR. Strontium is also much less active then calcium in regulating a second important calcium amplifying mechanism triggered by IP₃, a ubiquitous substance that also activates calcium release from the ER by an IP₃-specific receptor. At low concentrations of calcium, IP₃ acts as a potent stimulator of calcium release that acts to amplify the much smaller calcium influx during depolarization. When the calcium concentration is sufficiently elevated, calcium acts to inhibit further calcium release thus maintaining the calcium concentration within a limited concentration range. When strontium is present, it can mimic calcium in its ability to activate IP₃-induced calcium release, but strontium is not able to inhibit excessive calcium release causing both calcium and strontium to reach higher concentrations over an extended time. Strontium's ability to substantially inhibit calcium-induced release due to IP₃ is particularly important, since IP₃-induced calcium release is known to be responsible for generation of calcium waves. These types of strontium effects significantly change the calcium dynamics and calcium waveforms associated with neuropathic conditions, and thus contribute to strontium's suppressive effects on pain and pruritus.

### B. Strontium Inhibits Calcium-Dependent Neurotransmitter Release

While strontium also affects additional pathways that control the dynamics of calcium within nociceptors, there is one strontium-induced interference with calcium-dependent transmission of pain and itch-encoded calcium waves that is critically important for suppression of acute, chronic, and neuropathic conditions. That is, the ability of strontium to bind and inactivate synaptotagmin-1, a molecule that is principally responsible for neurotransmitter release in the DRG. Other members of the synaptotagmin molecular superfamily and related calcium-regulated molecules regulate the release of inflammatory neuropeptides, including substance P from the peripheral portion of a TCN in the epithelium. Substance P is known to be the most important inflammatory neuropeptide released from TCNs that activates virtually every inflammatory immune “white blood cell” (WBC), including mast cells that contain histamine and over 50 different inflammatory chemicals, including tumor necrosis factor-alpha (TNF-alpha), interleukin 1 alpha and beta (IL-1 alpha and beta) and IL-6. These three pro-inflammatory cytokines are believed to be the “first responders” that directly activate TCNs to cause pain and/or itching and are thought to be significant contributors to the development and maintenance of neuropathic conditions, as well as most skin conditions that are associated with inflammation, pain or itching.

Synaptotagmin-1 is a protein present on the surface of vesicles that contain and ultimately release neurotransmitters that bind to the post-synaptic neurons in the DRG and the peripheral TCN endings in the epithelium that relay the pain and itch-encoded signals to the brain. Normally, the frequency of the presynaptic neurotransmitter release from nociceptors are precisely matched so that the intensity, timing and other properties of the original pain or itch signal encoded in the calcium wave is accurately transmitted to the brain. The delay between the arrival of the calcium wave, neurotransmitter release and post-synaptic activation is usually about 1/1000^{th} of a second and the amount released is related to the intensity of the original TCN signal. This type of neurotransmission is termed “synchronous release,” since the timing of the arrival of the calcium wave is tightly synchronized to the release of neurotransmitters that triggers post-synaptic activation of the DRG nerve. Without this precise coupling, the frequency encoded pain or itch signal becomes distorted and garbled.

When strontium substitutes for calcium, the amplitude of synchronous neurotransmitter release in response to TCN activation is typically reduced by more then 90%. Strontium has an additional signal distorting effect that significantly distorts the timing of neurotransmitter release called “asynchronous release.” In contrast to synchronous release that is tightly coupled to the stimulating signal, asynchronous release may extend to several hundred milliseconds. With strontium, the total amount of neurotransmitter that is released may be the same as with calcium, however the strength of the synchronous release that contains the encoded pain or itch intensity information is strongly reduced, and the critical timing information is essentially destroyed. This strontium mechanism not only reduces the perceived severity of a pain or itch signal, but it also suppresses the release of substance P at the proximal end of the TCN in the epithelium at the original site of TCN activation. Strontium's ability to inhibit the release of TNF-alpha, IL-alpha and IL-6 is probably due to a similar interference of synaptotagmin or related calcium release mechanisms since it is the secretory mechanism used by virtually every cell. Suppression of synchronous neurotransmitter release also has an important therapeutic benefit for neuropathic pain or pruritus treatment.

Accordingly, in one embodiment, it is therefore desirable to further alter the calcium dynamics of nociceptors by further suppressing calcium release or by interfering with critical calcium-dependent pathways that are partially inhibited by strontium.

C. Strontium Binds to a Calcium-Sensing Receptor on Nociceptors that Suppresses Nociceptor Activation

Most, if not all, cells have a recently-identified surface receptor that detects the extracellular calcium concentration. Strontium also binds and activates the calcium-sensing receptor (CaSR) as efficiently as calcium, but triggers additional activities. This knowledge resulted in the commercial development of a simple strontium salt, strontium ranelate, an orally administered prescription drug for osteoporosis treatment in over 100 countries. Due to strontium's unique ability to mimic calcium's ability to activate the CaSR and, additionally, to activate additional pathways linked to the CaSR, strontium ranelate is the only known osteoporosis drug that has two independent osteoporosis therapeutic mechanisms—strontium inhibits bone loss by inhibiting bone-resorbing osteoclasts, and simultaneously stimulates osteoblasts that produce new bone.

Nociceptors also have a CaSR that inhibits nociceptor activation when the extracellular concentration of calcium is raised above normal, or if a similar concentration of strontium is administered. This mechanism is believed to contribute to the ability of strontium to rapidly inhibit TCN activation by, for example, highly acidic chemical peels such as 70% glycolic acid, pH 0.6, that cause burning pain within seconds after application. When strontium is mixed with the acid, burning pain and stinging is suppressed by 80% or more so that any remaining sensory irritation is not bothersome.

Activation of the CaSR also causes activation of several pathways that are known to increase both acute, chronic and neuropathic pain and pruritus and inflammation. Since in real world use, strontium typically inhibits pain and pruritus, it is likely that the pain and itch enhancing effect caused by activation of the CaSR by strontium is, in effect, negated by other strontium anti-irritant mechanisms. None the less, even a low level, “subclinical’ pain and itch-enhancing effect reduces the ability of strontium to effectively treat, prevent or reverse neuropathic conditions for which any excess TCN activation is known to promote the neuropathic condition.

Of particular concern is strontium's reported ability to bind to the CaSR and rapidly activate two of the MAPK molecules, p38 and ERK1/2, that are known to be among the primary contributors to peripheral and central nociceptor sensitization. Strontium binding to the CaSR is also reported to activate an important enzyme, phospholipase C, that produces two important regulatory molecules, the aforementioned IP₃, and diacylglycerol (DAG), both of which contribute to nociceptor activation and sensitization and inflammation. IP₃ is one of the most important and potent calcium releasing molecules that directly triggers calcium release from ER stores. Many of the pain and itch producing chemicals that are produced during inflammation, infection or trauma use the IP₃ pathway to activate nociceptors and produce the calcium waves that transmit pain and itch sensations. DAG is the principle activator of protein kinase C, a family of molecules that directly activates nociceptors and many of the pathways that produce pain and itch and inflammatory mediators. PKC is also known to be an important nociceptor sensitizer, since PKC inhibition can prevent or reverse neuropathic pain in animal models. PKC also activates NF-Kappa B, one of the most important stimulators of molecules that triggers pain, pruritus and inflammation and are thought to be able to directly cause neuropathic sensitization. It should be emphasized that the recognition that strontium produces its osteoporosis therapeutic benefits by binding to the CaSR is very recent and additional strontium-sensitive pathways will likely be identified. The fact that human nociceptors have the CaSR that regulate nociceptor activation suggests that the CaSR activation by topically-applied strontium may be working at a reduced level due to strontium's ability to inhibit important pain and itch pathways while simultaneously activating pathways via the CaSR that are known to trigger pain and itch pathways. Most importantly, since activation of these CaSR pathways are known to be important contributors to the development of neuropathic conditions, strontium's therapeutic potential may be substantially compromised.

### D. Strontium Blocks Endocytosis of Vesicles

Strontium also blocks rapid endocytosis of vesicles that are used to release both neurotransmitters and inflammation-induced molecules by blocking dynamin-1, a calcium-dependent GTPase. While not being bound by this or any particular mechanisms, it is believed that strontium's ability to block substance P release from Type C nociceptors and the pro-inflammatory cytokines TNF-alpha, IL-1 alpha and IL-6 is related to strontium's interference with such calcium-dependent release processes.

### E. Limitations of Strontium on Nociception and Inflammatory Pathways

It has been surprisingly discovered that the reason strontium is frequently unable to completely block pain, itching or inflammation is due to two factors: (1) the limited amount of strontium that can be topically applied, after which the hyperosmotic effects of the strontium salts themselves start to cause pain, itching or inflammation; and (2) the ability of strontium to stimulate pathways that may act to negate strontium's inherent anti-irritant activities, thus reducing the overall therapeutic benefit. Regarding the first factor, this is due to the fact that strontium has a relatively low potency in its ability to suppress pain, itching and inflammation compared to many other drugs with similar therapeutic goals. It is this low potency of strontium that prevents it from blocking pain when it is orally ingested in the form of the prescription drug, strontium ranelate that is approved for treatment of osteoporosis in over 100 countries.

Regarding the second factor, the degree to which strontium will negate its anti-irritant benefits depends on many factors related to the type of nerve damage that caused the neuropathic condition to develop (e.g., viral infection, physical trauma such as amputation or nerve compression, metabolic nerve damage as occurs in diabetes, coexisting inflammation and other factors.

For example, commonly used non-steroidal anti-inflammatory drugs (NSAID) like aspirin, ibuprofen and naproxen are typically used at oral doses of several hundred milligrams and provide an effective reduction of many types of inflammation-associated pain. Opioid pain relievers such as levorphanol, oxymorphone, oxycodone and hydrocodone are pharmacologically related to codeine, morphine and heroin and provide effective pain relief at oral doses in the range of 2 mg to about 10 mg per dose. In contrast, orally administered strontium salts such as strontium ranelate, an oral drug approved for the treatment and prevention of osteoporosis, is approved in over 100 countries, and is administered at a dose of 2,000 mg per day. Since strontium ranelate is a simple salt of strontium, it yields 680 mg of elemental strontium upon contact with water or gastric fluids. However, even at this high dose of pure elemental strontium, there are no reports of the ability to reduce pain or inflammatory reactions.

It has been determined that topical strontium has the ability to reduce pain, pruritus and inflammation due to the fact that topical formulations can deliver thousands of times higher strontium concentrations then can be achieved by oral, systemic administration. Even at the relatively high local concentrations that can be achieved topically when administered to the epithelium, the effect of strontium on key biochemical pathways that cause pain, pruritus and inflammation is only partial. For example, if the activity of a hypothetical pain or itch-producing pathway is inhibited by 90% to 100%, a patient reports that their pain or itching was completely stopped. In contrast, topically-applied strontium may only inhibit that pathway by 40% to 50%, sufficient inhibition for a patient to observe that the pain or itching was clearly reduced, but still present and still bothersome.

Strontium Affects Pathways Directly Associated with Gastroesophageal Reflux and Barrett's Esophagus

Previous studies have demonstrated that topically applied strontium can virtually abolish the intense burning pain caused by application of 70% glycolic acid, pH 0.6 to the skin. Similar pilot studies in humans also demonstrate strontium can inhibit burning pain due to installation of 1% lactic acid to the conjunctiva of the eye. Other published studies demonstrate that topically-applied strontium can inhibit pain and inflammation caused by a wide range of chemically and biologically-unrelated chemical irritants found in the environment, the workplace and the home and in foods.

Clinical observations also demonstrate that the pain of “sore throat” can be greatly relieved, if not eliminated within several minutes after gargling with a strontium chloride solution. Since acidic material from the stomach is what causes the burning pain and inflammation in GER, it is hypothesized that strontium would be effective in treating GER.

Unlike most antacids, which use acid neutralizing effects to reduce the acid-induced pain and inflammation, strontium acts directly on Type C nociceptors and reduces their sensitivity to acid-induced nerve depolarization that directly produces the sensation of GER-associated burning pain and also triggering inflammatory neuropeptide and pro-inflammatory cytokine release that is known to increase painful sensations by sensitizing Type C nociceptors.

Strontium can inhibit acid-induced inflammation by at least two independent anti-inflammatory mechanisms. In addition to acid-induced inflammation, strontium can inhibit inflammation caused by a broad range of chemically and biologically-unrelated inflammation irritating stimuli. Given the fact that the refluxed gastric juices and partly digested food contain many potentially irritating chemicals in addition to acid, strontium should provide a broad protective benefit to the esophagus.

### A. Strontium Inhibits Neurogenic Inflammation

Multiple studies have demonstrated that elevated IL-8 levels are associated with GER and may be pathogenically important as an inducer of chronic inflammatory damage to the esophagus found in GER. Substance P increases production of IL-8, a potent stimulator of neutrophil activation and a major contributor to the formation of reactive oxygen species (e.g. superoxide, hydrogen peroxide, hydroxyl radical, singlet oxygen) that are highly toxic and powerful inflammation inducers. Strontium's abilities to block substance P would prevent the elevation of IL-8 levels.

Additionally, recent studies recent studies suggest that elevated production of TNF-alpha, IL-1 alpha and IL-6 are important regulators and promoters of cancer metastasis. If these cytokines have a similar stimulatory effect on metastasis of esophageal adenocarcinoma, then strontium treatment may reduce metastatic disease from esophageal carcinoma, a frequently lethal result of chronic GER.

### B. Strontium Inhibits Acid Activated Inflammatory Pathways

As mentioned above, acid from the stomach activates many pathways that trigger pain and inflammation such as the release of substance P, neurokinin A, and CGRP. In unpublished animal studies, strontium has been shown to inhibit substance P release by Type C nociceptors, thus confirming human studies in which topical strontium application inhibited acid-induced pain and neurogenic inflammation. While the precise molecular mechanism by which strontium inhibits substance P release is unknown, it is believed that the release of substance P, neurokinin A and CGRP occur together and by the same or similar trigger mechanism. Thus, the fact that strontium inhibits substance P is a good indicator that strontium will also inhibited neurokinin A and CGRP.

### C. Additional Medical Benefit of Strontium

Osteoporosis is a chronic condition that results in bone weakening and loss of bone mass and density and results in increased fractures, especially in post-menopausal women, people over 70 and those taking certain drugs like oral anti-inflammatory glucocorticoids like prednisone, barbiturates, some antiepileptic drugs, L-thyroxine, cancer drugs like methotrexate, depot progesterone, anticoagulants like Heparin, warfarin and related coumarins, thiazolidinediones used for diabetes treatment, lithium therapy for psychiatric disorders and proton pump inhibitors, the most commonly used GER treatment drugs. Unfortunately the population that is most likely to use many of the above drugs are those most at risk for osteoporosis, older people.

In addition to a variety of prescription drug used to treat osteoporosis, calcium supplementation is widely recommended to help reduce bone loss. Since calcium supplementation provides limited benefits, additional non-toxic treatments or supplements would be of great benefit.

The strontium in the present disclosure can provide a substantial anti-osteoporosis benefit since strontium is the only substance known to stimulate new bone formation while suppressing bone loss. The prescription drug strontium ranelate is the only strontium drug approved for osteoporosis treatment and at its recommended daily dose delivers 680 mg of elemental strontium per day, of which approximately 25% is absorbed by the GI tract into the blood. Simple orally-administered strontium salts including those used in the present disclosure deliver the same rate of strontium absorption as strontium ranelate and for this reason strontium dietary supplements are widely available without a prescription in the United States. It is one object of the present disclosure to deliver a protective amount of elemental strontium, up to about 680 mg of elemental strontium per day to help prevent osteoporosis-induced bone loss. It is another object of the present disclosure to deliver additional calcium in the form of a calcium salt along with strontium to provide further osteoporosis protection, especially for older patients and those who ingest drugs that promote bone loss.

### Polyhydroxyphenols Affect Nociception and Inflammation Pathways

Polyhydroxyphenols are phenolic compounds possessing at least two hydroxyl groups. In one embodiment, the polyhydroxyphenols also exhibit one or more carboxyl groups. Also contemplated by the present disclosure are polymeric phenolic compounds that have two or more aromatic rings that typically, but do not necessarily have the same structure.

Polyhydroxyphenols act at different steps in the same inflammatory pathways inhibited by strontium, and thus in effect amplify the basic anti-irritant activity and nociceptor-protective activities of strontium. The effects of polyhydroxyphenols on some of the key nociception and inflammatory pathways are discussed below.

A. Polyhydroxyphenols Inhibit Multiple Inflammatory Pathways that Activate Nociceptors and Amplify Those of Strontium

Polyhydroxyphenols, in particular those that contain the gallic acid molecular structure, are powerful antioxidants that directly bind to components of NF-Kappa B and cause a direct inhibition of activation. They also directly inactivate superoxide, hydrogen peroxide, hydroxyl radicals and hypochlorous acid, thus preventing them from shifting the intracellular GSH concentration from being reduced, which activates NF-Kappa B and other redox activated inflammatory regulatory molecules and molecules that directly activate nociceptors. Polyhydroxyphenols also inhibit the expression of multiple cellular adhesion molecules like ICAM-1, VCAM-1 and members of the Selectin adhesion molecules that enable neutrophils and monocytes to extravasate from blood vessels and accumulate at sites of inflammation, thus contributing to nociceptor activation.

Polyhydroxyphenols are also inhibitors of protein kinase C (PKC) isozymes, and in particular, PKC epsilon. (See, for example, Cancer Res. 70(6): 2415-2423 (2010); and Biochem. Pharmacol. 38: 1627-1634 (1989), both incorporated by reference herein.) Also as described and demonstrated by both of these articles, methods for determining the degree of inhibition of PKC by compounds are known in the pharmaceutical arts. This is particularly useful since strontium can mimic the effects of calcium as a cofactor for PKC. As used herein, the polyhydroxyphenol will be considered to be a PKC inhibitor if it suppresses 10% or more of the activity of the PKC.

Polyhydroxyphenols are also known as adenosine triphosphate (ATP) analogues. ATP is a molecule that binds to the active sites of kinases like protein kinase C and other regulator kinases that are part of signal transduction pathways that active multiple inflammatory pathways, activate NF-Kappa B and directly activate nociceptors. These kinases are also known to be necessary for development of neuropathic nociceptor sensitization and neuropathic pain and pruritus. Polyhydroxyphenols that have hydroxyl groups adjacent to each other on the phenolic moiety in meta and para positions mimic the three dimensional structure of ATP and compete with ATP for the protein kinase ATP binding site. Binding of the polyhydroxyphenol to the ATP binding site prevents protein kinase from being active. Studies of the activities of various ATP analogues, such as flavonoids, are known in the literature. (See, for example, Phytochemistry Reviews 1:325-332 (2002), wherein the effect of flavonols on ATP-dependent activities was studies, incorporated by reference herein.)

Polyhydroxyphenols also posses an ability to inhibit the Fenton Reaction by which low concentrations of ferrous iron (Fe₂⁺⁺) and copper (Cu⁺⁺) catalytically produce the highly toxic and inflammatory hydroxyl radical that is a powerful inflammation activator.

Polyhydroxyphenols additionally are powerful inhibitors of prostaglandins and leukotrienes, particularly PGE₂. PGE₂ is one of the most important nociceptor sensitizers that is synthesized in virtually all inflammatory conditions.

Polyhydroxyphenols also have powerful inhibitory activities on one of the most important inflammatory molecules, the mast cell. Mast cells are present in the dermis and submucosal tissues throughout the body and are among the most important sources of preformed inflammatory mediators like histamine, TNF-alpha, IL-1, and IL-6. Nociceptors are activated, either directly or indirectly, by TNF-alpha, IL-1, IL-6 and others. Nociceptor activation is also a major stimulator of substance P release from TCN that directly activates mast cells, neutrophils and every other type of inflammatory white blood cell.

Furthermore, polyhydroxyphenols also have a critical ability to inhibit several inflammatory and nociceptor activating pathways that are stimulated by strontium. In particular, strontium's ability to activate the calcium-sensitive receptor (CaSR) on cells, including nociceptors is known to activate protein kinase A, protein kinase C and NF-Kappa B. Activation of each of these molecules is known to contribute to nociceptor activation and neuropathy development. Combining strontium with polyhydroxyphenols would limit such activation, thus negating the undesirable activities of strontium.

Polyhydroxyphenols, such as gallic acid, also have the ability to alter the intracellular calcium dynamics. Specifically, they reduce the increase of intracellular calcium in response to pain and inflammation-triggering extracellular stimuli.

Combining strontium with the polyhydroxyphenols as described herein, results in complexes that are more efficient inhibitors of many of the same nociceptor-activating pathways that are inhibited by strontium alone by having multiple, overlapping and distinct mechanisms. Additionally, polyhydroxyphenols also inhibit strontium-activated pathways that contribute to pain, pruritus and development of neuropathic diseases.

### B. Polyhydroxyphenols Bind to Conserved Hydrophobic Sites

Polyhydroxyphenols are known to bind to hydrophobic amino acids like phenylalanine, tyrosine and tryptophan via pi-pi bond stacking. The hydroxy groups are also important since they can hydrogen bond to the amide and carbonyl groups of a peptide backbone in a protein and to select amino acid side chains. Among amino acids to which gallic acid and other polyhydroxyphenols bind, proline and other aromatic amino acids are among the most important.

### C. Exemplary Polyhydroxyphenols

While polyhydroxyphenols are generally known to affect inflammatory pathways, many of which trigger nociception, not all polyhydroxyphenols are capable of interacting on all inflammatory pathways. Furthermore, structural similarity does not allow one to accurately predict which polyhydroxyphenols will work on which pathways. For example, caffeic acid blocks the calcium-activated potassium ion channel K_{Ca} 3.1. Gallic acid, which is structurally similar to caffeic acid, for example, does not block the K_{Ca} 3.1, the blocking of which would increase stomach acid secretion.

An exemplary polyhydroxyphenol in the practice of the present disclosure is gallic acid (3,4,5-trihydroxybenzoic acid) and derivatives of gallic acid. Gallic acid, and similarly structured polyhydroxyphenols, has multiple anti-inflammatory, antioxidant and inflammatory cell inhibitory activities that amplify the strontium regulated pathways that lead to suppression of nociceptor activation. Gallic acid also has a carboxylic acid group, which may be esterified with a sugar moiety such as glucose. For example, pentagalloyl glucose consists of five gallic acid residues that are esterified to one glucose molecule. This molecule will be cleaved in vivo by non-specific esterases, which free the individual gallic acid residues. Another example is tannic acid, which is a high molecular weight gallic acid polymer in which one or more gallic acid residues are esterified to a central glucose molecule. Yet another example is ellagic acid, which is a gallic acid dimmer While this molecule no longer possesses the gallic acid-like phenolic structure, it does maintain many of the same bioactivities of gallic acid and is thus useful in the practice of the present disclosure.

The polyhydroxyphenolic structure of gallic acid, tannic acid, ellagic acid, pentagalloyl glucose and similar polyhydroxyphenolic antioxidants provide such molecules with a number of important properties that both inhibit nociceptor-activating pathways suppressed by strontium and provide specific abilities to bind to and suppress several important kinases that are known to be important for development of neuropathic pain and pruritus conditions.

Gallic Acid and Related Molecules Affect Pathways Directly Associated with Gastroesophageal Reflux and Barrett's Esophagus

Gallic acid and related molecules such as tannic acid, ellagic acid, pentagalloyl glucose, have demonstrated affects on several molecular pathways directly associated to GER. Select molecular pathways are discussed below.

### A. Gallic Acid Inhibits Acid Induced Pain

Clinical studies have demonstrated that topically applied gallic acid substantially suppresses acid-induced pain in human clinical studies. Briefly, in a double blind study, 25% glycolic acid solutions with or without 250 millimolar of gallic acid were applied to an 2×8 inch area of the forearm of the study participants. The pain level was assessed at time of application and every minute thereafter for 10 minutes. Study participants rated the pain levels on a 0-4 scale where 0 was no pain; 1 was slight pain; 2 was mild pain; 3 was moderate pain; and 4 was unbearable pain. The results indicated that gallic acid was effective in reducing or preventing the acid induced pain.

### B. Gallic Acid Affects Acid Sensitive Ion Channels

Acid-sensitive ion channels (ASIC) are acid sensitive cationic channels present on Type C nociceptor and inflammatory cells. ASICs are activated by protons (H+). In GER, members of the ASIC superfamily are believed to trigger the pain and contribute to the subsequent neurogenic inflammation. Without wishing to be bound by any theory, it is believed that gallic acid directly binds to ASIC, and thus blocks their activity. However, this has not been proven at this time. By blocking ASIC mediated pain and inflammation, gallic acid would reduce the pain and inflammation that occurs in GER.

### C. Gallic Acid Reduces Stomach Acid Production

Parietal cells are the stomach epithelial cells that secrete gastric acid and intrinsic factor. These cells secrete acid in response to three types of stimuli: (1) histamine, which stimulates histamine H2 receptors; (2) acetylcholine, which stimulates M3 receptors; and (3) gastrin, which stimulates CCK2 receptors. It is believed that Gallic acid will potentate the therapeutic benefit of histamine H2 receptor antagonists by inhibiting biochemical pathways triggered by histamine in the acid-secreting parietal cells of the stomach. Such activity will be amplified by the demonstrated ability of strontium to inhibit activation of Type C nociceptors induced by injected histamine in human clinical studies. Human clinical studies by Maibach and coworkers demonstrated that topical strontium significantly inhibited injected histamine-induced itching.

### D. Gallic Acid Increases Expression of the Cystine/Glutamate Antiporter

Antiporters are proteins that move two different molecules in opposite directions across a membrane. The cystine/glutamate antiporter (System X_{c}⁻) causes the uptake of extracellular cystine in exchange for intracellular glutamate. As mentioned above, cystine is used to synthesize glutathione, which is essential for cellular protection from oxidative stress.

Gallic acid greatly increases the expression of the cystine/glutamate antiporter by increasing the nuclear levels of the transcription factor, nuclear factor-E2-related factor-2 (Nrf2) that is an antioxidant response element binding protein (ARE) that increases synthesis of proteins that protect against oxidative cellular stress. Increased expression of Nrf2 also directly increases expression of the cystine/glutamate antiporter thus allowing increased cystine penetration into a cell thus amplifying the overall therapeutic GER benefit of this ingredient combination.

### E. Gallic Acid Inhibits Histone Acetyltransferases

Histone acetyltransferases (HAT) are enzymes that acetylate conserved lysine amino acids on histone proteins. DNA is wrapped around histones, and by transferring an acetyl group to the histones, genes can be turned on and off Gallic acid is a broad and potent inhibitor of HAT Inhibition of HAT results in decreased gene expression, including expression of NF-kappa B, the master gene regulator of inflammation. Gallic acid also decreases expression and release of other inflammatory proteins such as TNF-alpha, IL-1 alpha and IL-6 that are known to be important contributors to GER-related inflammation and pain.

### F. Gallic Acid has Anti-Cancer Effects in Esophageal Cell Lines

If left untreated, chronic GER can develop into to esophageal cancer. In a recent study, gallic acid demonstrated a significant inhibition of cell proliferation in a series of cancer cell lines and induced apoptosis in esophageal cancer cells (TE-2) but not in non-cancerous cells (CHEK-1). (See Faried, et al., Int J Oncol. 2007 March; 30(3):605-13, herein incorporated by reference.) Such selective inhibitor activity against esophageal cancer cells suggests that gallic acid in combination with the ingredients of the present disclosure will provide a protective benefit for Barrett's esophagus, a pre-cancerous condition that may evolve into esophageal cancer.

G. Gallic Acid has Antimicrobial Effects on Helicobacter pylori, a Common Bacterium that Induces Gastric Acid Secretion and Gastric Ulcers

Peptic ulcers are areas of mucosal erosions in the gastrointestinal tract. Most peptic ulcers occur in the stomach (gastric ulcers) and first part of the small intestine (duodenal ulcers). A major causative factor of gastric and duodenal ulcers is chronic inflammation due to Helicobacter pylori that colonizes the gastric antral mucosa. Peptic ulcers produce similar symptoms to GER and in many cases are self-treated as GER. In a recent study gallic acid and cinnamic acid demonstrated ulcer-preventing properties as well as inhibition of H. pylori growth. (See Nanjundaiah, et al., Evid Based Complement Alternat Med. 2011;2011:249487;Epub 2011 Jun. 23, herein incorporated by reference.)

### Cysteine Based Antioxidants Affect Nociception and Inflammation Pathways

Cysteine is abbreviated by the three letter amino acid code, Cys. Cysteine has a thiol side chain, which is easily oxidized. Because of its high reactivity, the thiol group of cysteine has numerous biological functions.

A. Cysteine Based Antioxidants Inhibit Multiple Strontium Regulated Inflammatory Pathways that Activate Nociceptors

Cysteine is the rate-limiting amino acid that controls the synthesis of reduced glutathione (GSH). Accordingly, administration of a cysteine based antioxidant increases the concentration of GSH and reduces the intracellular concentration of oxidized glutathione (GSSG), thus normalizing a nociceptor's redox state. This has the immediate effect of inhibiting the activation of NF-Kappa B and the activation of many other redox-sensitive inflammatory pathways, thus reducing nociceptor activation by both direct and indirect pathways. Cysteine based antioxidants also have a unique antioxidant activity due to their thiol (SH groups) that suppress the ability of nitric oxide to covalently bond to and activate inflammatory kinases that are known to directly contribute to neuropathic conditions. Cysteine based antioxidants also directly inactivate other oxidants that activate inflammatory pathways and, most importantly, they inhibit nociceptor activation.

Due to its thiol group, cysteine based antioxidants also have the ability to directly bind to the thiol groups of cysteine residues within molecules that are part of inflammatory pathways that contribute to nociceptor activation. One particularly important thiol-sensitive pain-and inflammation-inducing molecule present on nociceptors and inflammatory cells is the transient receptor potential ankyrin, subtype 1 (TRPA1) that is highly sensitive to oxidation of its free cysteine amino acids that trigger both pain, itch and inflammatory responses. TRPA1 is unique among the known oxidation-sensitive ion channels in its sensitivity to a wide range of chemical irritants found in the environment, inflammatory chemicals released in inflammatory reactions like hydrogen peroxide and prostaglandin metabolites and chemicals in spicy, pungent foods. It is considered to be one of the most important “chemosensors” present on nociceptive neurons, immune cells and epithelial cells. Simple thiol anti-oxidants, like those in the present disclosure, can prevent or reverse oxidation of cysteines in TRPA1 and can thus prevent its activation and generation of pain and inflammatory responses due to irritant chemicals in the stomach contents and inflammatory reactions in the esophageal mucosa. Since there are many thiol-sensitive regulatory molecules, cysteine based antioxidants have the ability to block oxidation of critical cysteines in such molecules and thus block activation that leads to increased inflammation and nociceptor activation. For many redox sensitive cysteine regulated pathways, the concentration of calcium within nociceptors is increased and, as for many other nociceptor activators, the resultant calcium-concentration encoded pain, pruritus and activation signals contribute to the formation and the long-term continuation of neuropathic conditions. By blocking such cysteine oxidation induced calcium release, cysteine-based anti-oxidants contribute to strontium's inherent ability to similarly inhibit calcium-encoded signals, but by non-strontium mechanisms. By blocking calcium-dependent signals with distinct mechanisms, the overall nociceptor inhibitory activity is increased.

Cysteine based antioxidants also have the ability to alter the intracellular calcium dynamics. Specifically, they reduce oxidized regulatory proteins that regulate intracellular calcium levels. When cells are exposed to inflammatory mediators, reactive oxygen species oxidize molecules in the endoplasmic reticulum that stores calcium and releases it into the cytoplasm in response to an initial calcium-mediated signal. These oxidized molecules increase the sensitivity of the calcium release mechanism and increases the magnitude of the signal, thus potentially increasing both pain and inflammatory responses.

### B. Exemplary Cysteine Based Antioxidants

Cysteine exists in two enantiomeric forms, designated ‘L-cysteine’ and ‘D-cysteine’, of which the L form is used in living organisms while the D form is not. While both the L and D forms are contemplated in the present disclosure, the L form is most preferred. In addition, both L-Cys and D-Cys can form disulfide bonds between the two thiol groups to form a ‘dimer’, literally a pair of Cys molecules. Such disulfide bonds occur in many proteins and play a critical regulatory role in biochemical pathways due to the ease of their reversible formation by oxidative processes and dissolution by reductive processes. By convention, a disulfide-bonded dimer of cysteine is termed cystine. Thus one cysteine molecule under appropriate reducing conditions or enzymatic processing will yield two cysteine molecules. Cystine can be formed from either two L-Cys molecules, two D-Cys molecules, or one L-Cys and one D-Cys molecules. Another exemplary cysteine-based compound is N,S-diacetylcysteine.

Cysteine Based Antioxidants Affect Pathways Directly Associated with Gastroesophageal Reflux and Barrett's Esophagus

The addition of cysteine based antioxidants would be expected to increase acid induced pain and inflammation since antioxidants greatly increase activation of acid sensitive ion channels that cause pain by allowing an increased calcium flow into cells. However, in the presence of strontium, a calcium analog that flows through these channel, antioxidants should substantially increase the amount of strontium that enters cells through these channels and there by produce a greater inhibition of pain and inflammation.

### A. Cysteine Based Antioxidants Maintain the Cellular Ratio of GSH and GSSG

In damaged tissue, the levels of oxidized glutathione (GSSG) increase resulting and the triggering of NF-kappa B. As discussed above, cysteine is the rate-limiting amino acid that controls the synthesis of reduced glutathione (GSH). Administration of a cysteine based antioxidant increases the concentration of GSH and reduces the intracellular concentration of GSSG, thus inhibiting the activation of NF-kappa B.

### B. Commercial Benefit of Cystine

All molecules with a free thiol (—SH) group like cysteine or N-acetylcysteine (NAC) have a terrible odor and taste that has been compared to raw sewage. This is due to the fact that the olfactory receptors of the human nose are exquisitely sensitive to free thiols. Numerous attempts have been made to mask this odor in molecules like cysteine and NAC but without success. Since the thiol group in cystine forms a disulfide-bond between two cysteine molecules, there is no odor that would otherwise limit or prevent an oral, chewable tablet or liquid from being used by consumers. After ingestion, cystine is reduced within cells into two cysteine molecules with their two free thiols and no objectionable odor or taste occurs, yet the full therapeutic benefit of cysteine and its subsequent transformation into glutathione may be experienced by the consumer.

### The Complexes of the Present Disclosure

The compositions and formulas of the present disclosure were formulated to perform several functions: (1) neutralize stomach acid, (2) block stomach acid production, (3) create a protective barrier in the esophagus, (4) create a protective barrier in the stomach (5) reduce pain, and (6) reduce inflammation. The composition and formulations achieve the above objectives by interacting on several different molecular pathways, thus creating a synergistic effect that greatly outperforms the actions of each component alone. The main components of the compositions and formulations are discussed below.

### A. Strontium

Strontium is present as a divalent cation. Strontium is designated by its commonly used atomic symbol, ‘Sr’ and is depicted below.

Strontium mimics the ability of calcium to pass through voltage dependent calcium channels and once inside cells, it competes with calcium for binding to calcium-dependent receptors.

In one embodiment, compounds of the present disclosure use an acid neutralizing salt of strontium. Non-limiting examples of such strontium salts include strontium carbonate, strontium bicarbonate, strontium hydroxide, strontium phosphate, and strontium citrate. In another embodiment the strontium salt is strontium carbonate, which acts to neutralize acid in a manner similar to calcium carbonate, the commonly used antacid in many products. This molecule has two distinct pharmacological activities. The carbonate portion or other acid neutralizing salt component serves as a conventional acid neutralizing agent and in the acid environment of the stomach it combines with acid to form water. The strontium ion, unlike calcium used in conventional antacids, inhibits multiple pain and inflammation pathways that are known to be significant contributors to GER pain and inflammation. Strontium has four broad classes of pharmacological activities that reduce GER associated pain and inflammation: (1) disruption of intracellular calcium dynamics and calcium wave propagation, (2) inhibition of neurotransmitter release, (3) inhibition of substance P release and resultant neurogenic inflammation, and (4) inhibition of pro-inflammatory cytokine release, each of which was discussed in detail above.

### B. Polyhydroxyphenols

Polyhydroxyphenols are phenolic compounds possessing at least two hydroxyl groups, preferably in the ortho and para positions. One exemplary compound is 3,4,5-trihydroxy benzoic acid, also called gallic acid. The term “polyhydroxyphenol” does not include carboxylic acids, such as ranelate.

The polyhydroxyphenol can be added to the compositions described herein in essentially purified form, or they can be added in the form of polyhydroxyphenol-containing plant extracts, such as green tea and soy extracts.

The flavonoids are polyphenolic compounds possessing 15 carbon atoms; two six-carbon benzyl rings that are usually joined together by a linear, saturated three carbon chain. Other flavonoids may consist of two benzyl rings joined together by a third 5- or 6-carbon ring structure. Flavonoids constitute one of the most characteristic classes of compounds in higher plants. Many flavonoids are easily recognized as the pigments in flowering plants.

The polyhydroxyphenol may also function as an antioxidant. For example, gallic acid is a tri-hydroxyphenolic structure that has antioxidant activity. The monomeric phenolic compounds include for example, gallic acid (3,4,5-trihydroxybenzoic acid) and caffeic acid. Both compounds have a carboxylic acid group, which may be esterified with a sugar moiety such as glucose. In the case of gallic acid, such esterification produces glucogallin. Other organic esters may also be effective, such as the ethyl ester of gallic acid, ethyl gallate, or the propyl ester of gallic acid, propyl gallate.

Also contemplated by the present disclosure are polymeric phenolic compounds that have two or more aromatic rings that typically, but do not necessarily have the same structure. One such example is reservatrol. Another is pentagalloyl glucose, which consists of five gallic acid residues that are esterified to one glucose molecule. This molecule will be cleaved in vivo by non-specific esterases, which free the individual gallic acid residues. The use of such forms of polyhydroxyphenolic compounds has the added advantage of lowering osmotic activity, since one molecule of pentagalloyl glucose produces one unit of osmotic activity, as compared to five units of osmotic activity produced by the use of five separate molecules of gallic acid.

Tannic acid is another example of a high molecular weight gallic acid polymer in which one or more esterified gallic acid residues are esterified to a central glucose molecule.

Ellagic acid is an example of a gallic acid dimmer While this molecule no longer possesses the gallic acid-like phenolic structure, it does maintain many of the same bioactivities of gallic acid and is thus useful in the practice of the present disclosure.

Compounds having a flavone backbone include, for example, quercetin, and epicatechin (EC) and derivatives thereof, such as epigallocatechin gallate (EGCG found in green tea), epigallocatechin (EGC) and epicatechin gallate (ECG).

In one particular embodiment, the polyhydroxyphenols that are useful also exhibit one or more carboxyl groups, such as gallic acid. The carboxyl group can serve as an additional counterion, and also assist in matrix formation with an optional polyanionic polymer.

In one embodiment, the polyhydroxyphenol is gallic acid (3,4,5-trihydroxybenzoic acid), which is a naturally occurring antioxidant present in tea and many foods. Gallic acid is a plant-derived phenolic anti-oxidant that is present at high concentrations in foods and is a constituent of the polyhydroxyphenolic flavonoid family. It possesses multiple pharmacological activities that inhibit pathways in GER that cause pain and inflammation. In a broad sense, gallic acid has multiple protective activities: (1) antioxidant activity, (2) inhibition of acid-induced activation of acid sensitive ion channels, (3) direct inhibition of regulatory molecules in pain and inflammation pathways, and (4) inhibition of gene expression of pain and inflammation-triggering molecules, each of which is discussed in detail above.

### C. Cysteine Based Antioxidant

Cysteine is a naturally occurring amino acid that is present in many foods and proteins. Cystine is amino acid compound made up of two cysteine molecules bound together by a single disulfide bond. In the extracellular environment, cystine is the predominant form and is the only form transported into cells by a specific amino acid exchanging molecule, the System X_{c}⁻ antiporter. This protein exchanges extracellular cystine for intracellular glutamic acid using the relative concentration gradient between the two as the source of transport energy. Within cells, the disulfide bond of cystine is reduced to form two molecules of cysteine, each possessing a free sulfhydryl group. Free cysteine is then incorporated into the tripeptide, glutathione, gamma-Glu-Cys-Gly. Glutathione is the most prevalent and important intracellular thiol anti-oxidant in all cells and acts as a reduction/oxidation ‘redox’ switch that directly or indirectly controls the expression of hundreds of regulatory molecules, many of which are potent pain and inflammation inducers. Free cysteine can also directly inactivate reactive oxygen species (ROS) that can inactivate regulatory proteins and, oxidize lipids and directly cause mutations in DNA that can lead to abnormal cellular growth and cancer. In a broad sense, Cystine and its metabolite cysteine possess the following broad pharmacological activities: (1) antioxidant activity, (2) direct regulation of redox-sensitive regulatory molecules, and (3) inhibition of intracellular calcium levels that triggering pain and inflammation, each of which is discussed in detail above.

### D. Polymers

### 1. Alginic Acid

Alginic acid is naturally-occurring polysaccharide obtained from brown seaweed. Structurally, it is a polyanionic linear copolymer of (1-4)-linked beta-D-mannuronic acid and alpha-L-glucuronic acid. Due to it's repeated carboxyl groups, alginic acid electrostatically binds to positively charged atoms, such as strontium and calcium, when the pH of the vehicle is above the pKa of the carboxyl groups (approximately 3-4) causing them to be negatively charged and able to bind to strontium and calcium. As the pH is decreased and approaches the pH of an empty stomach (1 or less), hydrogen ions will compete with strontium and calcium and will displace and free strontium and calcium. Alginic acid thus acts as a typical ion exchange column matrix. By using various mixtures of naturally occurring alginic acid polymers, the rate of strontium and calcium release as a function of pH and ionic strength of the vehicle can be adjusted to achieve release over an extended period of time.

Alginic acid and its salts are widely used in foods, cosmetics and in medical devices. The FDA has declared alginic acid GRAS (Generally Recognized as Safe). A similar safety classification exists in the European Union and other countries.

When consumed after a meal, alginic acid forms a “raft” that floats on the surface of the gastric pool, providing a physical barrier to the irritating and inflammation-producing effects of gastric acid, proteases and other chemical irritants that is unrelated to any acid neutralizing activity that may be present. Alginic acid has been recognized by the FDA as a safe ingredient for GER therapy and is currently available in an OTC antacid in the US and it is available as an OTC Drug in the European Union for GER treatment and esophageal protection.

Alginic acid also has bioadhesive properties when applied to mucosal membranes. When a patient consumes alginic acid, the bioadhesive properties “coat” the esophagus, which protects the esophagus from exposure to the acidic contents of the stomach.

### 2. Polyvinylpyrrolidone

Polyvinylpyrrolidone (PVP) is commonly used as an inert carrier of therapeutically active molecules. Due to the varying polar structure of the PVP polymer, it presents multiple, repeating sites to which atoms and molecules may bind via ionic forces. Upon subsequent exposure to ionic media, such as water, the bound substance may be released into the media over an extended period of time. Thus facilitating gradual release of the substance as a function of pH and other adjustable conditions, such as temperature, etc. As such, the PVP acts as a “molecular reservoir” providing for sustained release of therapeutic substances. The PVP polymer may be in its native form, or it may be chemically modified by derivatization and/or crosslinking to adjust the “releasing” properties of the polymer. In one embodiment, PVP is used as a carrier for gallic acid, related gallic acid-containing molecules or other polyhydroxyphenolic molecules.

PVP is used in foods, cosmetics and in medical devices. It is used as an excipient in FDA approved oral prescription drugs as a tablet binder.

### Optional Compounds

The following compounds are recognized in the art to relieve symptoms associated with GER or are use in the manufacture of prescription and OTC drugs for GER. These compounds may optionally be combined with the formulations described in this disclosure.

### A. Additional Acid Neutralizing Agents

Additional acid neutralizing agents may be added. Non-limiting examples of such acid neutralizing agents include calcium carbonate, sodium bicarbonate, sodium citrate, aluminum hydroxide, aluminum phosphate, magnesium hydroxide, magnesium carbonate, magaldrate, almagate, and hydrotalcite.

Although calcium carbonate is one of the most widely used acid neutralizing agents in OTC GER treatment drugs, calcium actually causes an increase in acid production in the stomach after repeated use and over time may exacerbate acid secretion if antacid treatment is temporarily stopped. Thus, using calcium actually causes a dependency on antacid use.

### B. Histamine H2 Receptor Antagonists

Histamine H2 Receptor antagonists (H2RA) are a class of drugs used to block the action of histamine on parietal cells, specifically the histamine H2 receptors, thus decreasing the production of acid by these cells. Non-limiting examples of H2RA include cimetidine, ranitidine, famotidine, and nizatidine.

### C. Proton Pump Inhibitors

Proton pump inhibitors (PPI) are the most potent acid blocking treatment available and are among the most frequently used drugs in the world. They work by blocking the hydrogen ion pump of the stomach's parietal cells and prevent hydrochloric acid secretion into the stomach's lumen.

One of the most serious adverse reactions of PPI is their ability to cause osteoporosis, especially when they are used over many years in older patient who have a uniquely high risk of developing osteoporosis. The strontium based compounds of the present disclosure with a PPI could help prevent the development of osteoporosis. Specifically, strontium ranelate, a salt of ranelic acid that contains two strontium atoms as the “active ingredient,” is reported to be the most effective osteoporosis treatment to date. Strontium ranelate has been shown to stimulate new bone formation and inhibit bone loss.

The recommended dose of strontium ranelate is 2 grams orally per day, which delivers 680 mg of elemental strontium. Clinical studies show that approximately 25% of ingested strontium is absorbed into the blood and is entirely responsible for the observed improvement of osteoporosis. In one embodiment, compositions of the present disclosure containing PPIs are formulated to deliver as much strontium as strontium ranelate.

### D. Other Pharmaceutical Excipients

The compounds of the present disclose may also be formulated with additional ingredients known in the pharmaceutical art to increase stability, increase disintegration of solid tablets, or increase customer appeal. Non-limiting examples possible excipients include binders, bulking agents, diluents, sweeteners, flavorants, lubricants, and colorants.

Suitable binders include but are not limited to starches (corn starch, wheat starch, modified corn or wheat starch, Starch 1500, or pregelatinized starch), polymers (polaxomer, polyethylene glycol, polymethacrylate, polyethylene oxide, sodium carboxymethylcellulose, polyvinyl alcohol, calcium polycarbophil, or combinations thereof), natural gums (pectin, gelatin, gum Arabic, acacia, carrageenan, guar gum or tragacanth), and low or medium viscosity cellulose derivatives (carbomer, hydroxypropylmethylcellulose, hydroxypropylcellulose, microcrystalline cellulose, carboxymethylcellulose, hydroxyethylcellulose, methylcellulose or combinations thereof).

Suitable sweeteners include but are not limited to those derived from natural sources such as sugar, confectionary sugar, powdered sugar, sucrose, dextrose, glucose, lactose, fructose, maltodextrin, sorbitol, mannitol, xylitol, polydextrose, erythritol, or combinations thereof. Additionally, sweeteners also include but are not limited to artificial sweeteners such as aspartame, sucralose, acesulfamine K, saccharin derivatives, or mixtures thereof.

### Formulation and Administration

The acid neutralizing strontium salt based compositions disclosed herein can be formulated into an easy to consume dose. In the one embodiment, the at least one acid neutralizing strontium salt, at least one polyhydroxyphenol, at least one polymer, and optional at least one cysteine based antioxidant are formulated as a solid. Non-limiting examples of solids includes lozenges, chewable tablets, effervescent tablets, and chewing gum. The tablets are manufactured using conventional techniques in the pharmaceutical industry such as wet granulation, spray drying, or roller compaction.

Wet granulation is a method in which the active ingredient is mixed with a binder and other excipients in a suitable granulator. A granulating solution such as water or a solution containing dissolved binder is added to the powder blend while mixing it thoroughly. This process allows the powder blend to become wet and agglomerate to form granules. These granules are then dried in a conventional tray drier or a fluid bed drier to obtain dry granules, which are then milled and screened to obtain granules with desirable particle size distribution. These granules are then mixed with additional ingredients such as diluents, bulk sweeteners, intense sweeteners, flavors, disintegrants, lubricants, anti-adherents, glidants etc., and compressed in to tablets.

Spray drying is another method to granulate powders to obtain spherical free flowing powders, which can be blended with various other excipients and compressed in to tablets. Typically in a spray drying operation, the active ingredient, binder and other desired excipients are suspended in water and sprayed using an atomizer in to the spray drier. The droplets so generated by the atomizer are dried to form granules, which can be screened and milled to obtain desired particle size. The granules can be further processed to obtain tablets as explained above.

Roller compaction is another method to manufacture granules. In this method dry blend of active ingredient(s), binder and other desired excipients are forced through a pair of rollers held under high pressure, where the powder compacts to form thin wafer like sheets, which are then milled and screened to obtain free flowing granules. Small amounts of water can be sprayed on to the powder blend prior to feeding in to the rollers, to enhance binding properties of ingredients in this process. The granules so obtained can be further processed to obtain tablets as explained above.

In another embodiment, the at least one acid neutralizing strontium salt, at least one polyhydroxyphenol, at least one polymer, and optional at least one cysteine based antioxidant are formulated as a liquid.

The acid neutralizing strontium salt based compositions disclosed herein are designed to be taken in either a preventative manner or in an as needed manner. For preventative use, the compositions would be taken after every meal, for example, up to three hours post meat, up to two hours post meal, up to one hour post meal, or up to 30 minutes post meal. For as needed use, the compositions would be taken upon the onset of symptoms.

### EXAMPLES

The exemplary formulations that follow describe combinations of at least one acid neutralizing strontium salt, at least one polyhydroxyphenol, at least one polymer, and optionally at least one cysteine based antioxidant that can be used to treat symptoms associated with GER, GERD, or Barrett's esophagus. The final formulation amount for each compound are those commonly used in the art for formulating OTC and prescription treatments for patients suffering from symptoms associated with GER, GERD, or Barrett's esophagus. The acid neutralizing strontium salts can optionally be used in similar concentration as used in strontium ranelate.

### A. Exemplary Formulation 1—Basic

Strontium carbonate, gallic acid, alginic acid, and PVP are combined. Additional excipients are added to make a chewable tablet.

### B. Exemplary Formulation 2—Basic Plus Additional Acid Neutralizer

Strontium carbonate, calcium carbonate, gallic acid, alginic acid, and PVP are combined. Additional excipients are added to make a chewable tablet.

### C. Exemplary Formulation 3—Basic Plus Additional Acid Neutralizer

Strontium carbonate, aluminum hydroxide, magnesium hydroxide, gallic acid, alginic acid, and PVP are combined. Additional excipients are added to make a chewable tablet.

### D. Exemplary Formulation 4—Basic Plus Histamine H2 Receptor Antagonist

Strontium carbonate, gallic acid, alginic acid, PVP, and cimetidine are combined. Additional excipients are added to make a chewable tablet.

### E. Exemplary Formulation 5—Basic Plus Proton Pump Inhibitor

Strontium carbonate, gallic acid, alginic acid, PVP, and omeprazole are combined. Additional excipients are added to make a chewable tablet.

### F. Exemplary Formulation 6—Basic Plus Cysteine Based Antioxidant

Strontium carbonate, gallic acid, cystine, alginic acid, and PVP are combined. Additional excipients are added to make a chewable tablet.

### G. Exemplary Formulation 7—Basic Plus Cysteine Based Antioxidant and Additional Acid Neutralizer

Strontium carbonate, calcium carbonate, gallic acid, cystine, alginic acid, and PVP are combined. Additional excipients are added to make a chewable tablet.

### H. Exemplary Formulation 8—Basic Plus Cysteine Based Antioxidant and Additional Acid Neutralizer

Strontium carbonate, aluminum hydroxide, magnesium hydroxide, gallic acid, cystine, alginic acid, and PVP are combined. Additional excipients are added to make a chewable tablet.

### I. Exemplary Formulation 9—Basic Plus Cysteine Based Antioxidant, Additional Acid Neutralizer, and Histamine H2 Receptor Antagonist

Strontium carbonate, aluminum hydroxide, magnesium hydroxide, gallic acid, cystine, cimetidine, alginic acid, and PVP are combined. Additional excipients are added to make a chewable tablet.

### J. Exemplary Formulation 10—Basic Plus Cysteine Based Antioxidant, Additional Acid Neutralizer, and Proton Pump Inhibitor

Strontium carbonate, aluminum hydroxide, magnesium hydroxide, gallic acid, cystine, omeprazole, alginic acid, and PVP are combined. Additional excipients are added to make a chewable tablet.

### K. Additional Exemplary Formulations

Tables 1 below lists the specific compounds that make up the following formulations based on at least one acid neutralizing strontium salt, at least one polyhydroxyphenol, and at least one polymer.

For table 1, the following numbers correspond to the listed ingredient: (1) strontium carbonate; (2) strontium bicarbonate; (3) strontium hydroxide; (4) strontium phosphate; (5) strontium citrate; (6) gallic acid; (7) tannic acid; (8) pentagalloyl glucose; (9) ellagic acid; (10) alginic acid; and (11) polyvinylpyrrolidone.

Table 1 below lists the specific combinations for formulations A-T (on the vertical axis) using ingredients 1-11 (on the horizontal axis).

TABLE 1

| | 1 | 2 | 3 | 4 | 5 | 6 | 7 | 8 | 9 | 10 | 11 |
|---|---|---|---|---|---|---|---|---|---|---|---|
| A | + | | | | | + | | | | + | + |
| B | + | | | | | | + | | | + | + |
| C | + | | | | | | | + | | + | + |
| D | + | | | | | | | | + | + | + |
| E | | + | | | | + | | | | + | + |
| F | | + | | | | | + | | | + | + |
| G | | + | | | | | | + | | + | + |
| H | | + | | | | | | | + | + | + |
| I | | | + | | | + | | | | + | + |
| J | | | + | | | | + | | | + | + |
| K | | | + | | | | | + | | + | + |
| L | | | + | | | | | | | + | + |
| M | | | | + | | + | | | | + | + |
| N | | | | + | | | + | | | + | + |
| O | | | | + | | | | + | | + | + |
| P | | | | + | | | | | + | + | + |
| Q | | | | | + | + | | | | + | + |
| R | | | | | + | | + | | | + | + |
| S | | | | | + | | | + | | + | + |
| T | | | | | + | | | | + | + | + |

For formulations A-T, one or more of the following acid neutralizing agents can optionally be added: calcium carbonate, sodium bicarbonate, sodium citrate, aluminum hydroxide, aluminum phosphate, magnesium hydroxide, magnesium carbonate, magaldrate, almagate, and hydrotalcite.

For formulations A-T, one or more of the following histamine H2 receptor antagonists can optionally be added: cimetidine, famotidine, nizatidine, and rantidine.

For formulations A-T, one or more of the following proton pump inhibitors can optionally be added: omeprazole, lansoprazole, rabeprazole, pantoprazole, and esomeprazole.

For formulations A-T, one or more of the following acid neutralizing agents can optionally be added calcium carbonate, sodium bicarbonate, sodium citrate, aluminum hydroxide, aluminum phosphate, magnesium hydroxide, magnesium carbonate, magaldrate, almagate, and hydrotalcite. Additionally, one or more of the following histamine H2 receptor antagonists can optionally be added: cimetidine, famotidine, nizatidine, and rantidine.

For formulations A-T, one or more of the following acid neutralizing agents can optionally be added calcium carbonate, sodium bicarbonate, sodium citrate, aluminum hydroxide, aluminum phosphate, magnesium hydroxide, magnesium carbonate, magaldrate, almagate, and hydrotalcite. Additionally, one or more of the following proton pump inhibitors can be added: omeprazole, lansoprazole, rabeprazole, pantoprazole, and esomeprazole.

### L. Additional Exemplary Formulations with Cysteine Based Antioxidant

Table 2 below lists the specific compounds that make up the following formulations based on at least one acid neutralizing strontium salt, at least one polyhydroxyphenol, at least one cysteine based antioxidant, and at least one polymer.

For table 2, the following numbers correspond to the listed ingredient: (1′) strontium carbonate; (2′) strontium bicarbonate; (3′) strontium hydroxide; (4′) strontium phosphate; (5′) strontium citrate; (6′) gallic acid; (7′) tannic acid; (8′) pentagalloyl glucose; (9′) ellagic acid; (10′) cystine; (11′) N,S diacetylcysteine; (12′) alginic acid; and (13′) polyvinylpyrrolidone.

Table 2 below lists the specific combinations for formulations A′-AN' (on the vertical axis) using ingredients 1-13 (on the horizontal axis)

TABLE 2

| | 1′ | 2′ | 3′ | 4′ | 5′ | 6′ | 7′ | 8′ | 9′ | 10′ | 11′ | 12′ | 13′ |
|---|---|---|---|---|---|---|---|---|---|---|---|---|---|
| A′ | + | | | | | + | | | | + | | + | + |
| B′ | + | | | | | | + | | | + | | + | + |
| C′ | + | | | | | | | + | | + | | + | + |
| D′ | + | | | | | | | | + | + | | + | + |
| E′ | + | | | | | + | | | | | + | + | + |
| F′ | + | | | | | | + | | | | + | + | + |
| G′ | + | | | | | | | + | | | + | + | + |
| H′ | + | | | | | | | | + | | + | + | + |
| I′ | | + | | | | + | | | | + | | + | + |
| J′ | | + | | | | | + | | | + | | + | + |
| K′ | | + | | | | | | + | | + | | + | + |
| L′ | | + | | | | | | | + | + | | + | + |
| M′ | | + | | | | + | | | | | + | + | + |
| N′ | | + | | | | | + | | | | + | + | + |
| O′ | | + | | | | | | + | | | + | + | + |
| P′ | | + | | | | | | | + | | + | + | + |
| Q′ | | | + | | | + | | | | + | | + | + |
| R′ | | | + | | | | + | | | + | | + | + |
| S′ | | | + | | | | | + | | + | | + | + |
| T′ | | | + | | | | | | + | + | | + | + |
| U′ | | | + | | | + | | | | | + | + | + |
| V | | | + | | | | + | | | | + | + | + |
| W | | | + | | | | | + | | | + | + | + |
| X′ | | | + | | | | | | + | | + | + | + |
| Y′ | | | | + | | + | | | | + | | + | + |
| Z′ | | | | + | | | + | | | + | | + | + |
| AA′ | | | | + | | | | + | | + | | + | + |
| AB′ | | | | + | | | | | + | + | | + | + |
| AC′ | | | | + | | + | | | | | + | + | + |
| AD′ | | | | + | | | + | | | | + | + | + |
| AE′ | | | | + | | | | + | | | + | + | + |
| AF′ | | | | + | | | | | + | | + | + | + |
| AG′ | | | | | + | + | | | | + | | + | + |
| AH′ | | | | | + | | + | | | + | | + | + |
| AI′ | | | | | + | | | + | | + | | + | + |
| AJ′ | | | | | + | | | | + | + | | + | + |
| AK′ | | | | | + | + | | | | | + | + | + |
| AL′ | | | | | + | | + | | | | + | + | + |
| AM′ | | | | | + | | | + | | | + | + | + |
| AN′ | | | | | + | | | | + | | + | + | + |

For formulations A′-AN′, one or more of the following acid neutralizing agents can optionally be added: calcium carbonate, sodium bicarbonate, sodium citrate, aluminum hydroxide, aluminum phosphate, magnesium hydroxide, magnesium carbonate, magaldrate, almagate, and hydrotalcite.

For formulations A′-AN′, one or more of the following histamine H2 receptor antagonists can optionally be added: cimetidine, famotidine, nizatidine, and rantidine.

For formulations A′-AN′, one or more of the following proton pump inhibitors can optionally be added: omeprazole, lansoprazole, rabeprazole, pantoprazole, and esomeprazole.

For formulations A′-AN′, one or more of the following acid neutralizing agents can optionally be added calcium carbonate, sodium bicarbonate, sodium citrate, aluminum hydroxide, aluminum phosphate, magnesium hydroxide, magnesium carbonate, magaldrate, almagate, and hydrotalcite. Additionally, one or more of the following histamine H2 receptor antagonists can optionally be added: cimetidine, famotidine, nizatidine, and rantidine.

For formulations A′-AN', one or more of the following acid neutralizing agents can optionally be added calcium carbonate, sodium bicarbonate, sodium citrate, aluminum hydroxide, aluminum phosphate, magnesium hydroxide, magnesium carbonate, magaldrate, almagate, and hydrotalcite. Additionally, one or more of the following proton pump inhibitors can be added: omeprazole, lansoprazole, rabeprazole, pantoprazole, and esomeprazole.

The embodiments discussed above are provided to give those of ordinary skill in the art a complete disclosure and description of how to make and use the embodiments of the methods, and are not intended to limit the scope of what the inventor regards as his disclosure. Modifications of the above-described modes (for carrying out the disclosure that are obvious to persons of skill in the art) are intended to be within the scope of the following claims. All publications, patents, and patent applications cited in this specification are incorporated herein by reference as if each such publication, patent or patent application were specifically and individually indicated to be incorporated herein by reference.

1. A composition for treating gastroesophageal reflux comprising:
(a) at least one acid neutralizing strontium salt selected from the group consisting of: strontium carbonate, strontium bicarbonate, strontium hydroxide, strontium phosphate, and strontium citrate;
(b) at least one polyhydroxyphenol selected from the group consisting of: gallic acid, tannic acid, pentagalloyl glucose, ellagic acid, and esters thereof; and
(c) at least one polymer selected from the group consisting of alginic acid, polyvinylpyrrolidone, and xanthan gum.

2-18. (canceled)

19. A composition for treating gastroesophageal reflux comprising:
(a) at least one acid neutralizing strontium salt selected from the group consisting of:
strontium carbonate, strontium bicarbonate, strontium hydroxide, strontium phosphate, and strontium citrate;
(b) at least one polyhydroxyphenol selected from the group consisting of: gallic acid, tannic acid, pentagalloyl glucose, ellagic acid, and esters thereof;
(c) at least one cysteine based antioxidant selected from the group consisting of cystine and N,S-diacetylcysteine; and
(d) at least one polymer selected from the group consisting of alginic acid, polyvinylpyrrolidone, and xanthan gum.

20-42. (canceled)

43. The composition of claim 1, further comprising at least one additional acid neutralizing agent.

44. The composition of claim 43, wherein the acid neutralizing agent is selected from the group consisting of calcium carbonate, sodium bicarbonate, sodium citrate, aluminum hydroxide, aluminum phosphate, magnesium hydroxide, magnesium carbonate, magaldrate, almagate, and hydrotalcite.

45-55. (canceled)

56. The composition of claim 1, further comprising a histamine H2 receptor antagonist.

57. The composition of claim 56, wherein the histamine H2 receptor antagonist is selected from the group consisting of cimetidine, famotidine, nizatidine, and ranitidine.

58-61. (canceled)

62. The composition of claim 1, further comprising a proton pump inhibitor.

63. The composition of claim 62, wherein the proton pump inhibitor is selected from the group consisting of omeprazole, lansoprazole, rabeprazole, pantoprazole, and esomeprazole.

64-71. (canceled)

72. A method of treating heartburn or gastroesophageal reflux disease comprising administering the composition of claim 1 to a patient.

73. The method of claim 72, wherein the composition is administered to the patient after eating but before onset of gastroesophageal reflux symptoms.

74-75. (canceled)

76. The method of claim 72, wherein the composition is administered to the patient upon presentation of gastroesophageal reflux symptoms.

77. The composition of claim 19, further comprising at least one additional acid neutralizing agent.

78. The composition of claim 77, wherein the acid neutralizing agent is selected from the group consisting of calcium carbonate, sodium bicarbonate, sodium citrate, aluminum hydroxide, aluminum phosphate, magnesium hydroxide, magnesium carbonate, magaldrate, almagate, and hydrotalcite.

79. The composition of claim 19, further comprising a histamine H2 receptor antagonist.

80. The composition of claim 79, wherein the histamine H2 receptor antagonist is selected from the group consisting of cimetidine, famotidine, nizatidine, and ranitidine.

81. The composition of claim 19, further comprising a proton pump inhibitor.

82. The composition of claim 81, wherein the proton pump inhibitor is selected from the group consisting of omeprazole, lansoprazole, rabeprazole, pantoprazole, and esomeprazole.

83. A method of treating heartburn or gastroesophageal reflux disease comprising administering the composition of claim 19 to a patient.

84. The method of claim 83, wherein the composition is administered to the patient after eating but before onset of gastroesophageal reflux symptoms.

85. The method of claim 83, wherein the composition is administered to the patient upon presentation of gastroesophageal reflux symptoms.
